(19) United States
(12) Patent Application Publication
(10) Pub. No.: US 2018/0289541 A1
(43) Pub. Date: Oct. 11, 2018

(54) NOVEL DEVICE FOR ANESTHETIZING THE EYE

(71) Applicant: Envision Solutions LLC, Wenatchee, WA (US)

(72) Inventor: Michael McAllister, Wenatchee, WA (US)

(21) Appl. No.: 15/947,806

(22) Filed: Apr. 7, 2018

Related U.S. Application Data
(60) Provisional application No. 62/483,692, filed on Apr. 10, 2017.

(51) Int. Cl. A61F 9/00; A61M 19/00; A61F 13/15

(52) U.S. Cl. CPC A61F 9/0008; A61F 2013/15008; A61F 13/15; A61M 19/00

(57) ABSTRACT

Comfort packs and methods are provided for the delivery of drugs, especially liquid anesthetics, to the conjunctival fornix of the eye, using perilimbal anesthesia.

### FIELD OF THE INVENTION

The present invention relates to ocular anesthesia, particularly perilimbal ocular anesthesia, and to kits and methods for administering such anesthesia.

### BACKGROUND OF THE INVENTION

In recent years, advances in cataract surgery have led to greater levels of refractive precision, faster visual rehabilitation, and improved comfort and safety. Refinements in phacoemulsification techniques and intraocular lens (IOL) technology deserve much of the credit for these advances, but innovations in anesthesia, especially topical anesthesia, have also played an important role in improving outcomes and hastening visual recovery.

While topical anesthesia is favored by many surgeons in the majority of their cases, proper patient screening and careful preoperative planning are necessary in order to choose the best anesthesia for an individual patient. Mastery of all of the available techniques—intracameral, topical, parabulbar (sub-Tenon's), peribulbar, and retrobulbar anesthesia—along with an understanding of their advantages and disadvantages, is necessary in order to provide the highest level of care for all patients.

Topical anesthesia avoids the systemic and ocular risks of the previously described modalities. In addition, it allows for quick visual recovery. Monitored anesthesia care can be used, but surgery can also be performed without intravenous agents. Multiple agents are available for topical anesthesia and include tetracaine 0.5% drops, Tetravisc™ 0.5% gel (Ocusoft™, Richmond, Tex.), lidocaine 2% jelly, Xylocaine™ 4% (AstraZeneca, Wilmington, Del.), and bupivacaine 0.75%. Topical agents are administered at least 5 to 10 minutes prior to surgery.

Drop preparations are generally administered in two to three repeated doses separated by 5 to 10 minutes. Gel preparations have the benefit of coating the eye without requiring repeated doses. However, if used prior to dilating agents, gels can interfere with absorption. Therefore, many surgeons administrer a liquid preparation such as proparacaine 0.5% or tetracaine drops first and then complete the dilation protocols. After the pupil is dilated and 5 to 10 minutes prior to entering the operating room, Tetravisc™ or lidocaine gel can be topically administered to the eye. Lidocaine gel can be more viscous and at times more difficult to administer under the lids to anesthetize the superior and conjunctival conjunctiva and fornices. Tetravisc™ has an intermediate viscosity and therefore spreads like a liquid drop but also coats like a gel. Another flaw of these gel preparations is they can block the contact of antiseptics.

One other variant on this form of anesthesia includes soaking a sponge with both dilating and/or anesthetic drops (perilimbal anesthesia) and placing it in the conjunctival fornix for 10 to 15 minutes. However, the actual procedure of sponge placement can be more intrusive than drops alone, with sponges having to be cut to an appropriate size and shape and positioned in the conjunctival fornix.

Accordingly, it is an object of the present invention to provide methods and kits for topically anesthetizing the eye.

It is another object of the present invention to provide methods and kits useful for topically applying anesthesia to the eye and conjunctival fornix.

Still another object is to provide sterile kits for use in surgical theaters for delivering drugs including anesthesia to the eye and the interior fornix.

### SUMMARY OF INVENTION

A surprising versatile comfort pack for the delivery of drugs to the conjunctival fornix of the eye has been developed, particularly useful for the delivery of topical anesthetics. Thus, in a first principal embodiment the invention provides a method of administering perilimbal anesthesia to the conjunctival fornix of a patient in need thereof comprising: (a) providing a sterile sealed container in which is housed: (i) a plurality of sponges or cotton balls; (ii) a portion cup; and (iii) an applicator; (b) opening said container; (c) soaking one or more of said sponges or cotton balls with a liquid anesthetic in said portion cup to make a soaked sponge or cotton ball; (d) placing said soaked sponge or cotton ball onto the cornea of said patient; and (e) pushing said soaked sponge or cotton ball into the conjunctival fornix of said patient with said applicator.

The method is not limited to topical anesthetics, and can be used to deliver any drug to the conjunctival fornix of the eye. Thus, in a second principal embodiment, the invention provides a method of administering a drug to the conjunctival fornix of a patient in need thereof comprising: (a) providing a sterile sealed container in which is housed: (i) a plurality of sponges or cotton balls; (ii) a portion cup; and (iii) an applicator; (b) opening said container; (c) soaking one or more of said sponges or cotton balls with a liquid comprising said drug in said portion cup to make a soaked sponge or cotton ball; (d) placing said soaked sponge or cotton ball onto the cornea of said patient; and (e) pushing said soaked sponge or cotton ball into the conjunctival fornix of said patient with said applicator.

The invention further provides kits useful for practicing the methods of the current invention. Thus, in a third principal embodiment the invention provides a sterile sealed container in which is housed: (a) a plurality of sponges or cotton balls; (b) a portion cup; and (c) an applicator.

Additional advantages of the invention are set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawing, which is incorporated in and constitutes a part of this specification, illustrate several embodiments of the invention and together with the description serves to explain the principles of the invention.

FIG. 1 is a plane view of a transparent kit/comfort pack of the present invention depicting the various components thereof in a preferred container system.

FIG. 2 is a plane view of a transparent kit/comfort pack of the present invention depicting additional components in another preferred container system.

FIG. 3 is a side view of a drug dispensing device of the present invention.

FIG. 4 is a side view of a spheroidal sponge or cotton ball of the present invention.

FIG. 5 is an end view of a spheroidal sponge or cotton ball of the present invention.

FIG. 6 is a perspective view of an alternative design for the applicator of the present invention.

FIG. 7 is a side view of a sponge of the present invention attached to a pull string.

FIG. 8 depicts various alternative cylindrical designs for the sponge/pledgets of the present invention.

FIG. 9 depicts a side view of a preferred cotton ball of the present invention with a contiguous cotton tail.

### DETAILED DESCRIPTION

The present invention may be understood more readily by reference to the following detailed description of preferred embodiments of the invention and the explanation of the figure included herein.

### Definitions and Use of Terms

As used in this specification and in the claims which follow, the singular forms “a,” “an” and “the” include plural referents unless the context clearly dictates otherwise.

As used in this specification and in the claims which follow, the word “comprise” and variations of the word, such as “comprising” and “comprises,” means “including but not limited to,” and is not intended to exclude, for example, other additives, components, integers or steps. When an element is described as comprising a plurality components, steps or conditions, it will be understood that the element can also be described as comprising any combination of such plurality, or “consisting of” or “consisting essentially of” the plurality or combination of components, steps or conditions.

“Therapeutically effective amount” means that amount which, when administered to a human for supporting or affecting a metabolic process, or for treating or preventing a disease, is sufficient to cause such treatment or prevention of the disease, or supporting or affecting the metabolic process.

When ranges are given by specifying the lower end of a range separately from the upper end of the range, or specifying particular numerical values, it will be understood that a range can be defined by selectively combining any of the lower end variables, upper end variables, and particular numerical values that is mathematically possible. In like manner, when a range is defined as spanning from one endpoint to another, the range will be understood also to encompass a span between and excluding the two endpoints.

When used herein the term “about” will compensate for variability allowed for in the pharmaceutical industry and inherent in products in this industry, such as differences in product strength due to manufacturing variation and time-induced product degradation. The term allows for any variation which in the practice of good manufacturing practices would allow the product being evaluated to be considered therapeutically equivalent or bioequivalent in humans to the recited strength of a claimed product.

The phrase “acceptable” as used in connection with compositions of the invention, refers to molecular entities and other ingredients of such compositions that are physiologically tolerable and do not typically produce untoward reactions when administered to a subject (e.g., a mammal such as a human).

“Cotton” refers to any fibrous cellulosic material capable of being formed into a defined shape and absorbing aqueous liquid anesthetics.

The “fornix,” or “conjunctival fornix,” refers to the void between the loose arching folds connecting the conjunctival membrane lining the inside of the eyelid with the conjunctival membrane covering the eyeball.

### Discussion of Principal Embodiments

In a first principal embodiment the invention provides a method of administering perilimbal anesthesia to the conjunctival fornix of a patient in need thereof comprising: (a) providing a sterile sealed container in which is housed: (i) a plurality of sponges or cotton balls; (ii) a portion cup; and (iii) an applicator; (b) opening said container; (c) soaking one or more of said sponges or cotton balls with a liquid anesthetic in said portion cup to make a soaked sponge or cotton ball; (d) placing said soaked sponge or cotton ball onto the cornea of said patient; and (e) pushing said soaked sponge or cotton ball into the conjunctival fornix of said patient with said applicator.

In a second principal embodiment, the invention provides a method of administering a drug to the conjunctival fornix of a patient in need thereof comprising: (a) providing a sterile sealed container in which is housed: (i) a plurality of sponges or cotton balls; (ii) a portion cup; and (iii) an applicator; (b) opening said container; (c) soaking one or more of said sponges or cotton balls with a liquid comprising said drug in said portion cup to make a soaked sponge or cotton ball; (d) placing said soaked sponge or cotton ball onto the cornea of said patient; and (e) pushing said soaked sponge or cotton ball into the conjunctival fornix of said patient with said applicator.

In a third principal embodiment, the invention provides a sterile sealed container in which is housed: (a) a plurality of sponges or cotton balls; (b) a portion cup; and (c) an applicator.

### Discussion of Subembodiments

The contents of the container are not strictly limited to those recited in the principal embodiments, and can further include the liquid anesthetic or drug for use in the methods of the present invention. Thus, in another embodiment the container further houses a dropper filled with said liquid anesthetic. In one particular embodiment, the dropper comprises a squeezable tube having a void therein for housing said anesthetic, in fluid communication with an opening sealed by a removable cap.

The sponges or cotton balls form an integral part of the kits and methods of the present invention and can be further described based on their physical properties, their shape, their size, or their chemical composition. For example, they can be defined based on their capacity to absorb an aqueous composition (i.e. a composition that contains greater then 50, 60, 70 or 80 wt % water). In a preferred embodiment, the sponge or cotton ball is capable of absorbing greater than 15, 17, 19, 21, or even 23 times its weight of an aqueous composition.

The shape of the sponges or cotton balls can be selected based on the preferences of the operating physician, but are preferably standardized and customized for ready insertion into the conjunctival fornix. A preferred shape is roughly that of a spheroid (i.e. an elliptical sphere), having a ratio of minimum diameter to maximum diameter of from 0.2:1 or 0.5:1 to 1:1, with a 1:1 ratio defining a circular sphere. Given the nature of the material with which the sponge or cotton ball is constructed, it will be understood that the term “spheroid” or “elliptical sphere” refers to a shape that roughly resembles, or corresponds to, a strict mathematical spheroid or elliptical sphere. The maximum diameter of the spheroids preferably ranges from 1 to 8 mm, from 2 to 6 mm, from 3 to 5 mm, or even most preferably, about 4 mm.

Another preferred shape is a cylinder having an elliptical cross section. The cylinders preferably have a diameter:length ratio of from 0.1:1 or 0.3:1 to 1:1. The maximum length of the cylinders preferably ranges from 2 to 10 mm or from 3 to 8 mm.

Alternatively, the sponges/cotton balls can take the form of a pledget, i.e., a small flat compress made of cotton gauze, or a tuft of cotton wool, lint, or a similar synthetic material. The maximum diameter of the pledgets preferably ranges from 1 to 8 mm, from 2 to 6 mm, from 3 to 5 mm, or even most preferably, about 4 mm.

In a still further embodiment, the sponges/cotton balls are attached to a string so that, after the method of the present invention has been performed and the eye has been sufficiently anesthetized, the sponge/cotton ball can be removed from the fornix simply by pulling the string and pulling the sponge or cotton ball out of the eye.

The sponges/cotton balls are preferably comprised of a fibrous material such as cotton or other cellulosic material that is capable of absorbing relatively large quantities of liquid anesthetic. A particularly preferred material comprises cellulose fiber derived from natural or synthetic cotton. The plurality of cotton balls or sponges is preferably capable of absorbing from 0.5 to 5 ml, from 0.6 to 3 ml, from 0.8 to 2 ml, or about 1 ml of aqueous liquid.

In one particular embodiment, regardless of the shape of the sponge or cotton ball, a void runs through the length of the sponge or cotton ball. Thus, in one embodiment, the plurality of sponges or cotton balls comprise a length and a void extending the length of the sponge or cotton ball.

The container used in the present invention is not critical and can be constructed using any suitable design and construction material, such as a plastic tray or a bag, as long as the container defines an interior volume large enough to house the components, and an exterior surface. In a preferred embodiment, the contain is a sealed plastic bag impervious to external pathogens. When access to the interior components is desired, the bag can be cut apart using scissors, or a tearing mechanism can be built into the bag, whereby the two opposing plastic sheets are pulled apart, or a zipper is torn from the bag. The container is preferably sterile, in the sense that no pathogens are contained in the interior volume of the container, although there is no universal need for sterility on the outside.

In one preferred embodiment, one of the interior walls of the container is constructed with receptacles to receive one or more elements of the kit, such as the applicator, cup or dropper. Thus, in one particular embodiment, the sterile sealed contained comprises a wall in which is formed receptacles for receiving said portion cup and said dropper and said applicator.

The applicator preferably is a cotton tipped applicator. The cotton tip is preferably large enough for manipulating the sponges/cotton balls deep within the conjunctival fornix, but not so large as to cause excessive pressure against the eye or eyelid during insertion of the sponges/cotton balls. In one embodiment the cotton tip is in the shape of a wedge. The stick of the applicator will typically range between about 5 and 15 cm in length.

The kits and methods of the present invention can be used to administered practically any drug to the eye, but they are preferably used to administer aqueous drug solutions, and even more preferably used to administer aqueous anesthetic solutions. A preferred topical anesthetic is a 4% lidocaine solution. In a preferred embodiment, the methods and kits of the invention deliver from 0.1 to 5 ml of liquid anesthetic to the eye, from 0.25 to 3 ml, from 0.5 to 2 ml, or about 1 ml. In a particularly preferred embodiment, about 1 ml of a 4% lidocaine solution is administered by the methods and kits of the present invention.

### DESCRIPTION OF FIGURES

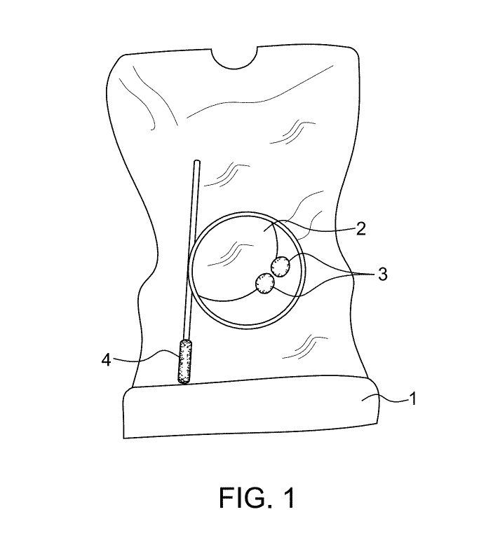

Turning to FIG. 1, an image of an exemplary kit of the present invention is provided which depicts a sealed container (“sterile packaging”) (1), in which is housed a portion cup (2) for soaking the sponges/cotton balls with a liquid solution, two cotton balls (3) approximately 4 mm in diameter, and a cotton tip applicator (4). The sealed container (1) is formed from two equally sized opposing sheets sealed around their perimeter. In practice, a user will open the container (1) by pulling the two opposed sheets apart. The sponges/cotton balls (3) are placed inside portion cup (2), and a liquid composition, such as 4% lidocaine, is then added to the portion cup and soaked into the sponges. The physician removes the soaked sponges/cotton balls (3) using a sterile instrument such as the cotton tip applicator or a surgical forceps, places the sponges/cotton balls (3) onto the human eye, and pushes or manipulates the sponges/cotton balls into the conjunctival fornix using the cotton tip applicator (4). After the eye is sufficiently anesthetized, the sponges/cotton balls can then be removed using the cotton tipped applicator or other suitable instrument.

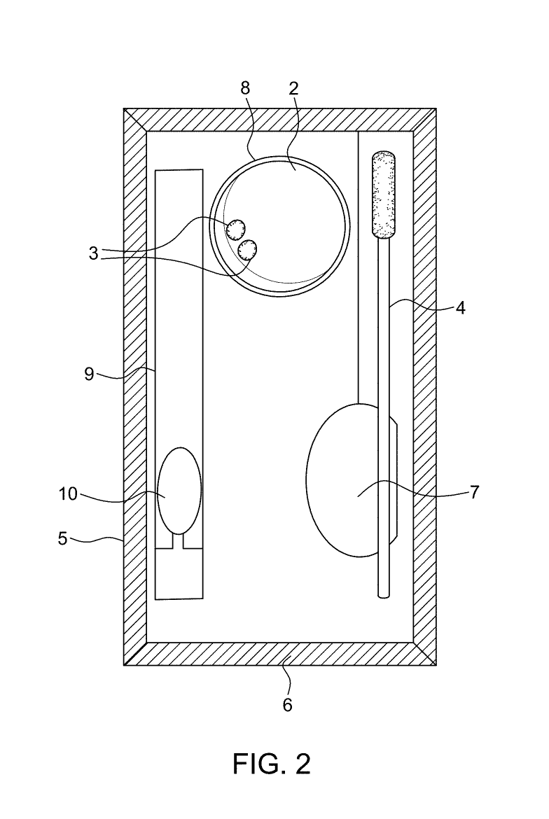

FIG. 2 depicts another exemplary kit of the present invention which depicts a sealed container (5) in which is housed the same portion cup (2), cotton balls (3), and cotton tip applicator (4). The container further includes a bottom wall (6) molded with a receptacle (7) for receiving applicator (4) and a receptacle (8) for receiving portion cup (2). The container also includes a receptacle (9) for receiving a dropper (10).

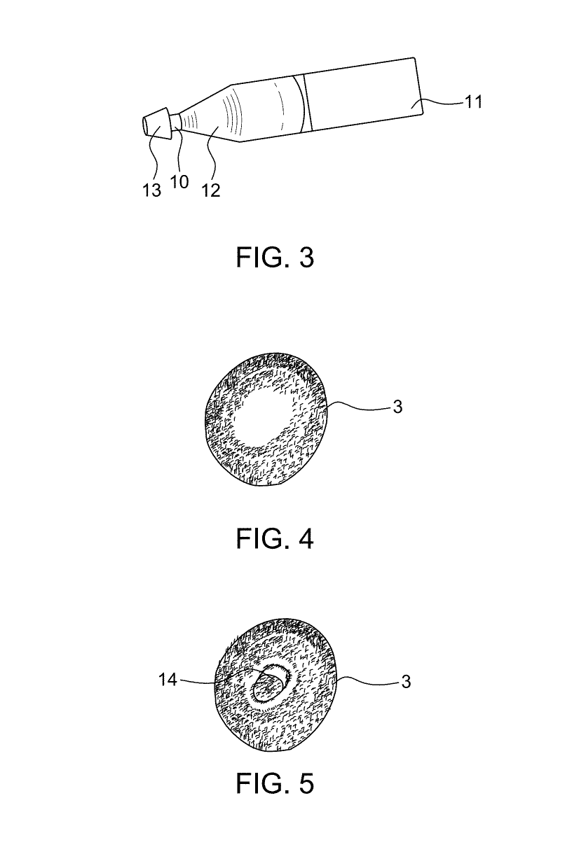

FIG. 3 gives a preferred construction for dropper (10). The wide end (11) of the dropper is preferably solid for grasping. The hollow end (12) forms a cavity in which the anesthetic or drug is stored. The tip through which the drug or anesthetic is dispensed is covered by a cap (13). FIGS. 4 and 5 depict cotton ball (3) from two separate perspectives. FIG. 4 depicts a side view of the cotton ball, whereas FIG. 5 depicts a view from the end of the cotton ball, illustrating a void (14) extending through the length of the cotton ball.

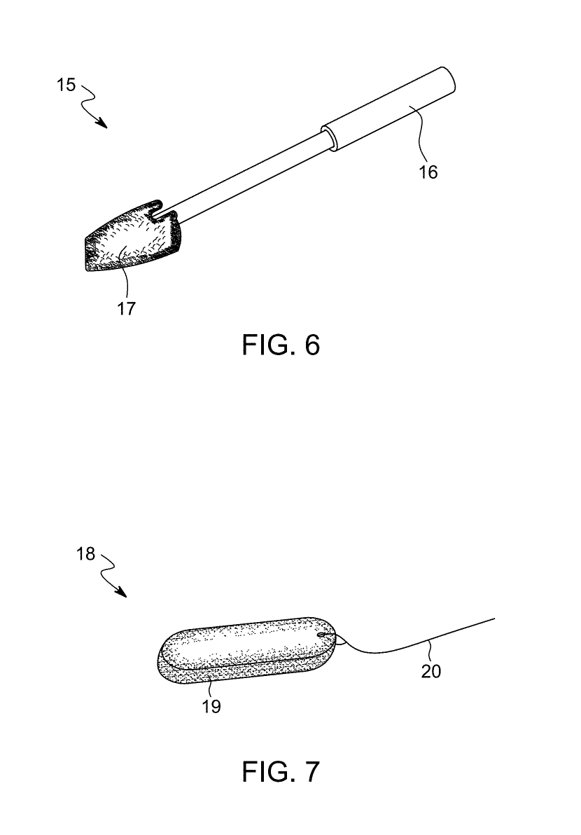

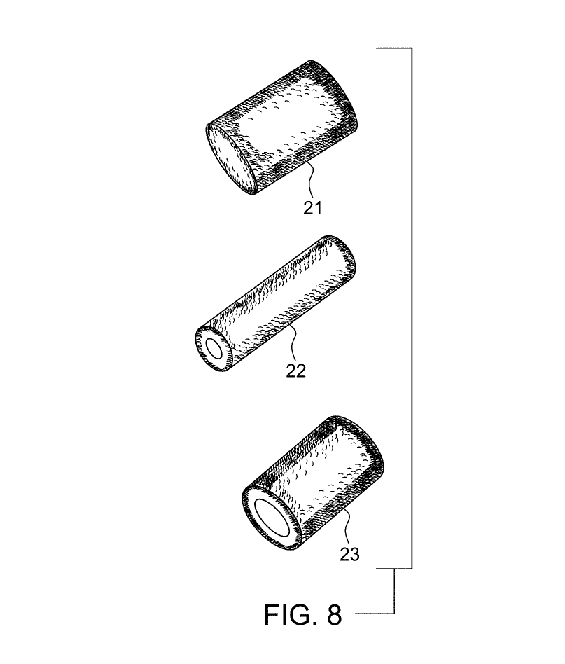

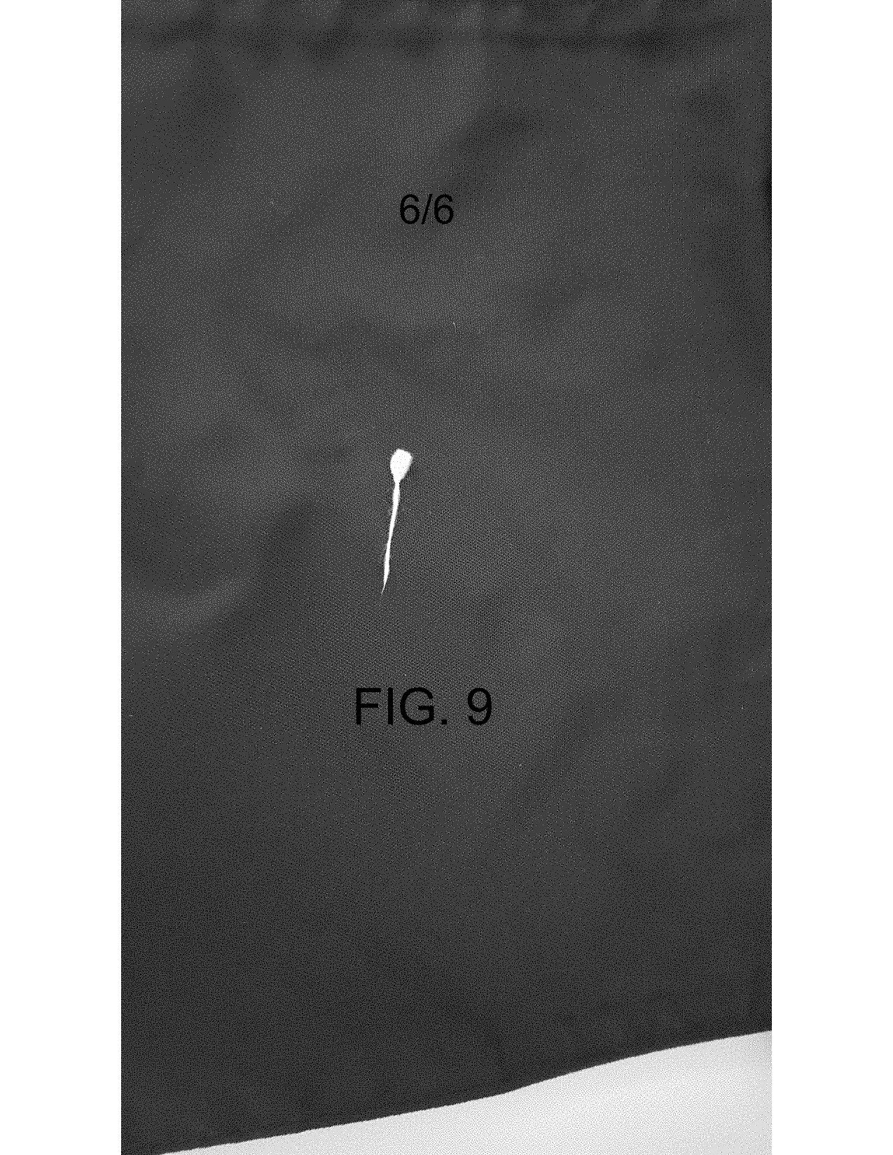

FIG. 6 depicts an alternative construction for the cotton tip applicator, wherein a wedge-shaped sponge tip (17) is attached to the end of a stick (16). FIG. 7 depicts a pledglet (18) that can act as the sponge or cotton ball of the present invention in the shape of a cylinder (19) attached to a string (20). FIG. 8 depicts several different cylindrical shaped sponges or cotton balls, numbered (21), (22) and (23). FIG. 9 depicts a cotton ball with a tail that is particularly useful in the methods and kits of the present invention. The ball and tail are formed of one contiguous fibrous structure. The cotton ball is spun from cotton yarn and the trailing thread is spun with the exact same yarn that is used to spin the ball. These shapes are given to provide greater context for the shapes that the sponge or cotton ball of the present invention can assume.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of administering perilimbal anesthesia to the conjunctival fornix of a patient in need thereof comprising:
a) providing a sterile sealed container in which is housed:
i) a plurality of sponges or cotton balls;
ii) a portion cup; and
iii) an applicator;
b) opening said container;
c) soaking one or more of said sponges or cotton balls with a liquid anesthetic in said portion cup to make a soaked sponge or cotton ball;
d) placing said soaked sponge or cotton ball onto the cornea of said patient; and
e) pushing said soaked sponge or cotton ball into the conjunctival fornix of said patient with said applicator.

2. The method of claim 1 wherein said sterile sealed container further houses a dropper filled with said liquid anesthetic.

3. The method of claim 2, wherein said dropper comprises a squeezable tube having a void therein for housing said anesthetic, in fluid communication with an opening sealed by a removable cap.

4. The method of claim 2 wherein said sterile sealed contained comprises a wall in which is formed receptacles for receiving said portion cup and said dropper and said applicator.

5. The method of claim 1, wherein said plurality of sponges or cotton balls predominantly comprise cellulose fibers.

6. The method of claim 1, wherein said plurality of sponges or cotton balls are in the shape of elliptical spheres having a maximal diameter of from 1 to 8 mm.

7. The method of claim 1, wherein said plurality of sponges or cotton balls are in the shape of cylinders having an elliptical cross section.

8. The method of claim 1, wherein said plurality of sponges or cotton balls comprise a length and a void extending the length of the sponge or cotton ball.

9. The method of claim 1, wherein said plurality of sponges or cotton balls comprise a tail selected from:
a) a contiguous thread of yarn spun from the same yarn as the cotton ball; or
b) a string attached to said sponge or cotton ball.

10. The method of claim 1, wherein said sponges or cotton balls comprise a dry weight, comprising soaking said sponges or cotton balls with said liquid anesthetic in an amount equal to at least 15 times the dry weight of the sponges or cotton balls.

11. The method of claim 1, wherein said liquid anesthetic comprises greater than 50% water.

12. The method of claim 1, wherein said method delivers from 0.5 to 5 ml of liquid anesthetic to the eye.

13. The method of claim 1, wherein said container is a plastic bag.

14. The method of claim 1, wherein said container has an interior volume and an exterior surface, said interior volume is sterile and said exterior surface is not sterile.

15. The method of claim 1, wherein said applicator is a cotton tipped applicator.

16. The method of claim 1, wherein said applicator comprises a wedge-shaped tip.

17. A method of administering a drug to the conjunctival fornix of a patient in need thereof comprising:
a) providing a sterile sealed container in which is housed:
i) a plurality of sponges or cotton balls;
ii) a portion cup; and
iii) an applicator;
b) opening said container;
c) soaking one or more of said sponges or cotton balls with a liquid comprising said drug in said portion cup to make a soaked sponge or cotton ball;
d) placing said soaked sponge or cotton ball onto the cornea of said patient; and
e) pushing said soaked sponge or cotton ball into the conjunctival fornix of said patient with said applicator.

18. The method of claim 17, wherein said plurality of sponges or cotton balls predominantly comprise cellulose fibers.

19. The method of claim 17, wherein said plurality of sponges or cotton balls are in the shape of an elliptical sphere having a maximal diameter of from 1 to 8 mm.

20. The method of claim 17, wherein said sponges or cotton balls comprise a dry weight, comprising soaking said sponges or cotton balls with said liquid in an amount equal to at least 15 times the dry weight of the sponges or cotton balls.

21. The method of claim 17, wherein said liquid comprises greater than 50% water.

22. The method of claim 17, wherein said method delivers from 0.5 to 5 ml of liquid to the eye.

23. The method of claim 17, wherein said container is a plastic bag.

24. The method of claim 17, wherein said container has an interior volume and an exterior surface, said interior volume is sterile and said exterior surface is not sterile.

25. The method of claim 17, wherein said applicator is a cotton tipped applicator.

26. A sterile sealed container in which is housed:
a) a plurality of sponges or cotton balls;
b) a portion cup; and
c) an applicator.

27. The container of claim 26 further housing a dropper filled with said liquid anesthetic.

28. The container of claim 27, wherein said dropper comprises a squeezable tube having a void therein for housing said anesthetic, in fluid communication with an opening sealed by a removable cap.

29. The container of claim 27 wherein said sterile sealed contained comprises a wall in which is formed receptacles for receiving said portion cup and said dropper and said applicator.

30. The container of claim 26, wherein said plurality of sponges or cotton balls predominantly comprise cellulose fibers.

31. The container of claim 26, wherein said plurality of sponges or cotton balls are in the shape of an elliptical sphere having a maximal diameter of from 1 to 8 mm.

32. The container of claim 26, wherein said sponges or cotton balls comprise a dry weight, and said sponges or cotton balls are capable of absorbing a liquid in an amount equal to at least 15 times the dry weight of the sponges or cotton balls.

33. The container of claim 26, further comprising a drug in a liquid comprising greater than 50% water.

34. The container of claim 26, wherein said sponges or cotton balls are capable of absorbing from 0.5 to 5 ml of liquid.

35. The container of claim 26, wherein said container is a plastic bag.

36. The container of claim 26, wherein said container has an interior volume and an exterior surface, said interior volume is sterile and said exterior surface is not sterile.

37. The container of claim 26, wherein said applicator is a cotton tipped applicator.
